(19) United States
(12) Patent Application Publication
(10) Pub. No.: US 2016/0008167 A1
(43) Pub. Date: Jan. 14, 2016

(54) CRYOTHERAPY DEVICE

(71) Applicant: Finn MAHLER, Vesenaz (CH)

(72) Inventor: Finn Mahler, Vesenaz (CH)

(21) Appl. No.: 14/772,855

(22) PCT Filed: Mar. 7, 2014
(86) PCT No.: PCT/IB14/59522

§ 371 Date: Sep. 4, 2015

(30) Foreign Application Priority Data
Mar. 8, 2013 (FR) 13 52088

(51) Int. Cl. A61F 7/02; A61F 13/02; A61F 13/00

(52) U.S. Cl. CPC A61F 7/02; A61F 13/00038; A61F 13/0273; A61F 2013/00187; A61F 2013/0028; A61F 2007/0219; A61F 2007/0222; A61F 2007/0231

(57) ABSTRACT

A cryotherapy device includes a flexible strip (1) having a receiving compartment (4) and a fixing section (5), and an insert having at least one chamber filled with a cooling fluid for maintaining a decrease in temperature and shaped in such a way as to be housed and held in the receiving compartment (4). The insert is a flexible silicone pouch filled with a thermal material gel able to maintain thermal cooling. The receiving compartment (4) is arranged on a first end of the fixing section (5) of the flexible strip (1). The flexible strip (1) is a self-adhesive strip having a length (L) longer than 1.0 m, which has self-attaching means for allowing the first surface of the strip to attach and adhere to the second surface of the strip.

### TECHNICAL FIELD OF THE INVENTION

The present invention relates to devices for emergency treatment of the trauma injuries and the contusions often sustained by the limbs and other parts of the body during various sports activities, work activities or travel. The invention is intended to apply in particular to the treatment of the human body.

It is known that such trauma injuries and contusions can be treated effectively by rest, cryotherapy, i.e. physical use of cold on the injured areas, compression of the injured areas and, if possible, elevation of the injured areas. These techniques are often designated by the acronym R.I.C.E. (Rest, Ice, Compression, Elevation).

Rest is prescribed because continued activity may result in additional damage to the injured area, thereby delaying the healing process, increasing pain, and stimulating internal bleeding.

Cold is prescribed because it causes vaso-constriction, which reduces effusion of blood into the injured area. The reduction in the subcutaneous temperature also modifies the conduction speed of the sensory nerves and in this way reduces the sensation of pain.

Compression also reduces bleeding and secondary oedema around the injured area and accelerates healing.

Elevating the injured area to a position above the heart reduces the blood pressure in the injured region, thereby reducing tissue haemorrhage while at the same time reducing pain.

Traditionally, cold has been applied by placing ice on the injured area. However, ice is a solid material that does not effectively conform to the shape of the surface of the body in the injured area.

More recently, it has been proposed that ice be replaced by a bag or container filled with a synthetic material with a high calorific power, by combining the bag or container with dressings for holding it in place in the injured area on the human body.

Thus, the document US 2012/0330202 A1 describes such a device specially adapted for application to the shoulder of a patient, the document U.S. Pat. No. 7,481,786 B2 describes such a device specially adapted for application around the ankle of a patient, the document U.S. Pat. No. 5,065,758 describes such a device specially adapted for application around the wrist of a patient, and the document U.S. Pat. No. 7,621,944 B2 describes such a device shaped like a sleeve and specially adapted for application around an intermediate joint of a limb.

Moreover, the document US 2009/0005718 A1 discloses such a device having an intermediate receiving compartment between two opposite fixing portions that extend, on either side respectively of the intermediate compartment, along a length of between 400 mm and 850 mm. A pouch made of vinyl, polypropylene or polyethylene and containing a gel of thermal material is accommodated and retained in the receiving compartment. The two fixing portions have, arranged and fixed to the end of their inner face, a hook-type fastening pad of a type known under the trademark VELCRO, which can fasten itself to all the outer faces of the fixing portions, which are themselves covered with complementary loops.

The devices described in the above documents are each clearly adapted for a particular use, and they do not each allow treatment, by cold and application of sufficient pressure, of most of the areas of the body that are liable to sustain injury during common activities. In particular, each of these devices is not capable of effectively treating very different areas such as an ankle, a knee, the back, a wrist, an elbow, a shoulder, or any kind of muscle lesion of the human body.

In addition, it appears that the devices described are not sufficiently deformable to ensure that the source of cold is applied satisfactorily to injured areas of the body that have pronounced curvatures. This results in inadequate treatment of these injured areas, since the devices do not conform precisely to the anatomical contours.

### SUMMARY OF THE INVENTION

It is an object of the present invention to avoid the disadvantages of these known devices by optimizing the application of cold to the area of the body to be treated, by ensuring sufficient pressure for satisfactory contact which promotes transmission of cold from the cold source to the area of the body to be treated, by ensuring a uniform distribution of the pressure in order to equalize the application of the cold across the area of the body to be treated, and by ensuring satisfactory comfort through a uniform distribution of the pressure of the device on those areas of the body that surround the area of the body to be treated, by conforming precisely to the anatomy.

For this purpose, its is necessary to permit sufficient deformation of the source of cold, such that it follows exactly the shape of the area to be treated, and, simultaneously with this flexibility, to ensure that the source of cold is maintained and compressed against the area to be treated, and to distribute, in adjustable and precise manner, the fastening and the tightening of the device.

The present invention also has the object of ensuring, with a single device, that the source of cold is correctly maintained in place and satisfactorily compressed regardless of the area of the body where injuries occur and are to be treated, namely the knees, the ankles, the wrists, the elbows, the shoulders, the back, a muscle lesion.

To achieve these objects and others, the present invention proposes a device for treatment by cold, comprising:

a flexible bandage with a first face and second face, having a receiving compartment and a fixing portion,

an insert which has a flexible pouch filled with a gel of thermal material able to maintain a reduction of the temperature, and which is shaped so as to be accommodated and retained in the receiving compartment,

and in which:

the flexible bandage comprises a single fixing portion,

the receiving compartment is arranged at a first end of the single fixing portion of the flexible bandage,

the flexible bandage is a self-adhesive bandage, of which the length is greater than 1.0 m and which has self-fastening means distributed so as to permit adherence of any part of a substantial portion of its first face to any part of a substantial portion of its second face.

A substantial portion of a face of the bandage is understood as meaning an area occupying all or most of the length of the bandage face starting from one of its ends. The substantial portions of the first and second faces of the self-adhesive bandage can advantageously occupy the entirety of said first and second bandage faces.

This combination of features permits:

by virtue of the flexibility of the flexible pouch, a suitable conformation to the shape of the surface of the body to be treated,

by virtue of the length and shape of the flexible bandage, fixing to any articulated area of the body,

by virtue of the self-adhesive nature of the bandage, suitable tightening regardless of the area of the body to be treated, ensuring satisfactory pressure between the receiving compartment and the area of the body to be treated, and ensuring a better comfort.

In this way, the device according to the invention has a better ability to cool the area of the body to be treated, adapts to all the areas of the body that are normally liable to sustain injury, and guarantees satisfactory comfort for the patient.

According to a preferred embodiment applicable to the treatment of the human body, provision is also made that the flexible bandage has a length of between 1.0 m and 3.5 m, preferably of between 1.7 m and 1.9 m. A length of over 1.7 m generally allows the flexible bandage to be wound several times on itself around the area of the body to be treated, so as to obtain an optimal compressive effect.

Such a range of lengths permits effective fixing and optimal tightening on all areas of the body that are liable to sustain injury.

Advantageously, the fixing portion of the flexible bandage can have a width of between 8 cm and 20 cm, preferably of between 12 cm and 15 cm.

In this way, the width is sufficiently great to distribute the pressure over an extensive surface of the body, and it is sufficiently small to adapt to the pronounced curvatures of certain areas of the body, thereby ensuring satisfactory comfort.

According to an advantageous embodiment, the receiving compartment is generally rectangular, with a width greater than the width of the fixing portion.

The rectangular shape of the receiving compartment makes it possible to use, as the insert with thermal fluid, a rectangular bag or container more commonly available on the market. At the same time, the greater width of the receiving compartment makes it possible to treat a sufficiently extensive surface around the injured area of the body, while maintaining the advantages associated with a relatively narrow flexible bandage.

In practice, good results can be obtained by using a receiving compartment having a width of between 24 cm and 30 cm, and a length of between 28 cm and 34 cm.

Preferably, the receiving compartment is offset laterally with respect to the fixing portion, such that a first lateral edge of the receiving compartment is in a continuation of a first lateral edge of the fixing portion.

In this way, a first half of the receiving compartment is effectively held by the adjacent part of the fixing portion of the flexible bandage, and the second half of the receiving compartment can be effectively held by another part of the fixing portion which covers it after it has been wound around the body to be treated.

The self-adhesive nature of the flexible bandage can advantageously be obtained by using a flexible bandage made of a textile material having in itself, on its first face, a distributed structure with multiple non-aggressive micro-loops and having, on its second face opposite the first face, a distributed structure of non-aggressive micro-hooks that are able to fasten themselves to said micro-loops.

This therefore prevents the introduction of a degree of rigidity, which is inevitably caused in the prior art by the attached pads or bandages of the type known under the trademark VELCRO, and their aggressive nature is also avoided.

Advantageously, the receiving compartment can be delimited, along an inner face of the flexible bandage, by a wall of netting which is rigidly connected along its peripheral edge to the flexible bandage, except for a free edge defining an opening which is provided for passage of the insert and which is oriented towards the fixing portion.

The wall of netting constitutes a permeable barrier, promoting the transfer of cold between the insert and the area of the body to be treated, while ensuring the presence of a separating gap in order to avoid burning the skin at the location of the insert.

The hold of the second end of the fixing portion of the flexible bandage can be improved by providing, at this second end, two transverse tongues extending away from each of its longitudinal edges.

According to an advantageous embodiment, the gel of thermal material is a gel based on propylene glycol maintaining its flexibility when cold, or a gel packaged in an insert having multiple compartments articulated with one another.

Better flexibility at low temperatures can be ensured by preferably using a flexible pouch made of silicone.

### BRIEF DESCRIPTION OF THE DRAWINGS

Other subjects, features and advantages of the present invention will become clear from the following description of particular embodiments and by reference to the attached figures, in which:

FIG. 1 is a plan view of the outer face of a device for treatment by cold according to one embodiment of the present invention;

FIG. 2 is a side view in longitudinal section along the axis I-I of FIG. 1;

FIG. 3 is a perspective view illustrating the first step in applying the device for treatment by cold of FIG. 1 to the knee of a patient;

FIG. 4 is a perspective view illustrating the device for treatment by cold of FIG. 1 when arranged on the knee of the patient;

FIG. 5 is a perspective view of the device for treatment by cold of FIG. 1 when arranged around the ankle of a patient;

FIG. 6 is a perspective view of the device for treatment by cold of FIG. 1 when arranged around the abdomen of a patient for the purpose of applying cold to the patient's back;

FIG. 7 is a perspective view illustrating the first step in applying the device for treatment by cold of FIG. 1 to the shoulder of a patient;

FIG. 8 is a perspective view illustrating the second step in applying the device for treatment by cold of FIG. 1 to the shoulder of the patient; and

FIG. 9 is a perspective view illustrating the final step in applying the device for treatment by cold of FIG. 1 to the shoulder of the patient.

### DESCRIPTION OF THE PREFERRED EMBODIMENTS

In the embodiment illustrated in the figures, the device for treatment by cold according to the invention comprises a flexible bandage 1, of elongate shape along a longitudinal axis I-I between a first end 2 and a second end 3.

The flexible bandage 1 comprises a receiving compartment 4 and a single fixing portion 5, the receiving compartment 4 forming the first end 2 of the flexible bandage 1 and thus being arranged at a first end of the single fixing portion 5 of the flexible bandage 1.

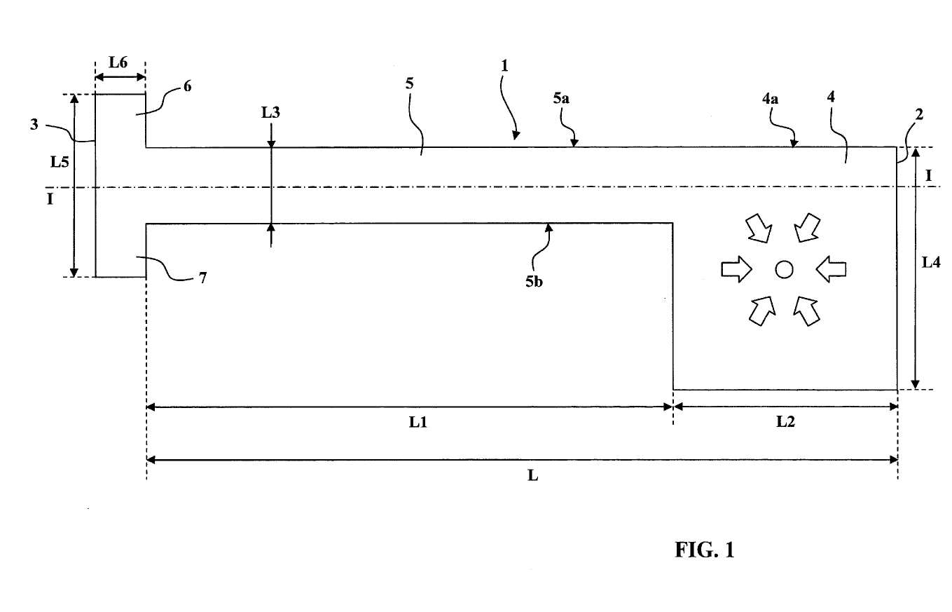

The flexible bandage 1 has a total length L sufficient to surround any part of the body for which treatment by cold may customarily be prescribed. A length of over 1.0 m may be suitable for most cases of treatment of the human body. In practice, it will be possible to choose a total length of between 1.0 m and 3.5 m, preferably of between 1.7 m and 1.9 m. In the example illustrated in FIG. 1, the total length L is about 1.81 m.

The fixing portion 5 of the flexible bandage 1 has a length L1 of about 1.5 m in the example illustrated in FIG. 1, while the receiving compartment 4 has a length L2 of between 28 and 34 cm, for example of about 31 cm in the embodiment illustrated in FIG. 1.

The fixing portion 5 of the flexible bandage 1 has a width L3 of between 8 cm and 20 cm, preferably of between 12 cm and 15 cm. In the embodiment illustrated in FIG. 1, the width L3 is about 13.5 cm.

The fixing portion 5 of the flexible bandage 1 can have a thickness of about 4 mm, although this thickness is not critical.

The receiving compartment 4 has a width L4 of between 24 and 30 cm. In the embodiment illustrated in FIG. 1, the width L4 is about 26 cm.

As will be seen from FIG. 1, the receiving compartment 4 is offset laterally with respect to the fixing portion 5, such that a first lateral edge 4a of the receiving compartment 4 is in a continuation of a first lateral edge 5a of the fixing portion 5.

Near the second end 3 of the flexible bandage 1, two transverse tongues 6 and 7 are shown, which extend away from each of the longitudinal edges 5a and 5b and have a length of about 5 cm and a width L6 of about 6 cm. The width L5 between the ends of the tongues 6 and 7 is therefore about 23 cm.

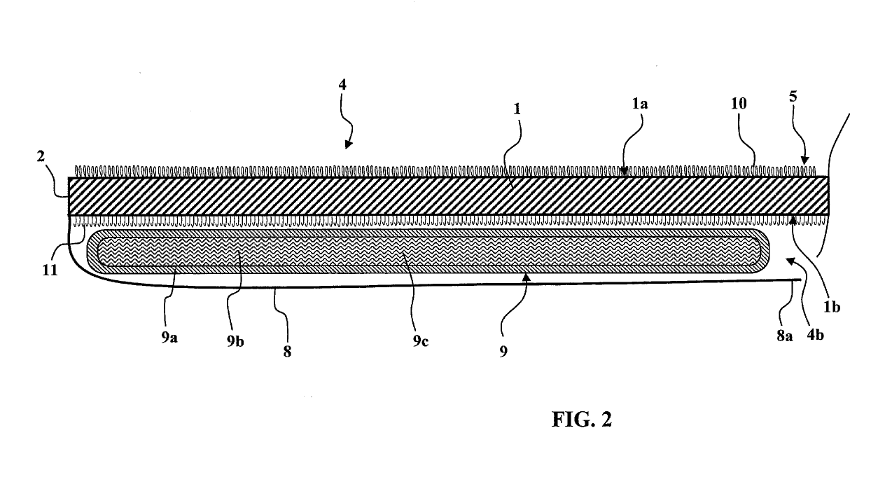

Reference will now be made to FIG. 2, which is a partial side view, in longitudinal section along the axis I-I of FIG. 1, near the first end 2 of the device.

FIG. 2 shows the receiving compartment 4 and a part of the fixing portion 5. The thickness of the flexible bandage 1 is shown, with an outer face la and an inner face 1b. The receiving compartment 4 is delimited, towards the outside, by the thickness of the flexible bandage 1, and is delimited, towards the inside, that is to say along the inner face lb of the flexible bandage 1, by a wall 8 of netting. The wall 8 of netting is made up of open meshes of synthetic material having a thickness and a mesh size that are suitable for promoting the transfer of cold while avoiding too direct a contact between the source of cold and the skin of the patient to be treated. Good results have been obtained, for example, with a wall 8 of netting made up of polyethylene filaments having a diameter of about 0.5 mm and a mesh size of about 3 mm.

The receiving compartment 4 must be able to contain and retain an insert 9 constituting the source of cold. For this purpose, the wall 8 of netting is rigidly connected along its peripheral edge to the flexible bandage 1, except for a free edge 8a defining an opening 4b for passage of the insert 9. The opening 4b of the receiving compartment 4 is preferably oriented towards the fixing portion 5.

The insert 9 is a flexible pouch which is delimited by a leaktight peripheral wall 9a made of silicone and defining a chamber 9b filled with a gel of thermal material 9c that is able to maintain a temperature.

The peripheral wall 9a of the flexible pouch can preferably be made of a silicone elastomer vulcanized at high temperature, with a thickness of about 0.7 mm, defining a generally flat chamber that measures about 900 cm³.

The gel of thermal material 9c can advantageously be a gel based on propylene glycol, which maintains its flexibility at a low temperature. An example of an effective gel is a mixture of propylene glycol, a food dye, distilled water, an antiseptic, cellulose for maintaining viscosity at ambient temperature, and a sodium salt for maintaining plasticity at low temperature. Thus, the gel can be packaged in a flexible pouch with a single compartment.

Alternatively, the gel of thermal material 9c can be a gel that becomes more rigid when cold but is contained in a wall 9a of an insert 9 with multiple compartments that are articulated with one another in order to ensure sufficient flexibility.

Advantageously, it will be possible to use a bag from the range designated by the brand name BLUE ICE from the company RUBBERMAID.

The flexible bandage 1 is a self-adhesive bandage having fastening means that are distributed so as to allow adherence of its first face 1a to its second face 1b, in this case to allow adherence of its outer face 1a to its inner face 1b. The fastening can advantageously be ensured regardless of which zone of the outer face 1a and of the inner face 1b is concerned.

In practice, it will advantageously be possible to use a flexible bandage 1 made of a textile material which itself has, distributed across its first face, such as the outer face 1a, a structure with multiple non-aggressive micro-loops illustrated by reference sign 10 in FIG. 2, the textile material itself having, distributed on its second face, in this case on the inner face 1b, a structure with non-aggressive micro-hooks illustrated by reference sign 11 in FIG. 2, the micro-hooks 11 being able to fasten themselves to the micro-loops 10. A description of such a flexible bandage made of a textile material with non-aggressive micro-loops and micro-hooks will be found in the document U.S. Pat. No. 7,231,789 B2. By contrast to the attached structures of pads or bandages with multiple hooks and multiple loops known under the trademark VELCRO, which are relatively rigid and aggressive when they are in contact with the skin, the structures of textile material with micro-hooks and micro-loops are flexible and not aggressive, such that they are tolerated by the skin and promote the distribution of pressure on the body to be treated.

This results in automatic fastening that is entirely controllable along the full length of the flexible bandage 1, permitting optimal tightening of the device on the body of a patient and ensuring maximum comfort.

Alternatively, it will be possible to provide fastening means that are distributed across only a substantial part of the length of the flexible bandage 1, on condition that this substantial part is sufficiently long to still allow the tightening to be controlled. For example, it will be possible for micro-loops to be arranged along half the length of the first face la of the fixing portion of the bandage near its first end, and it will be possible for micro-hooks to be arranged along half the length of the second face lb of the fixing portion near its second end.

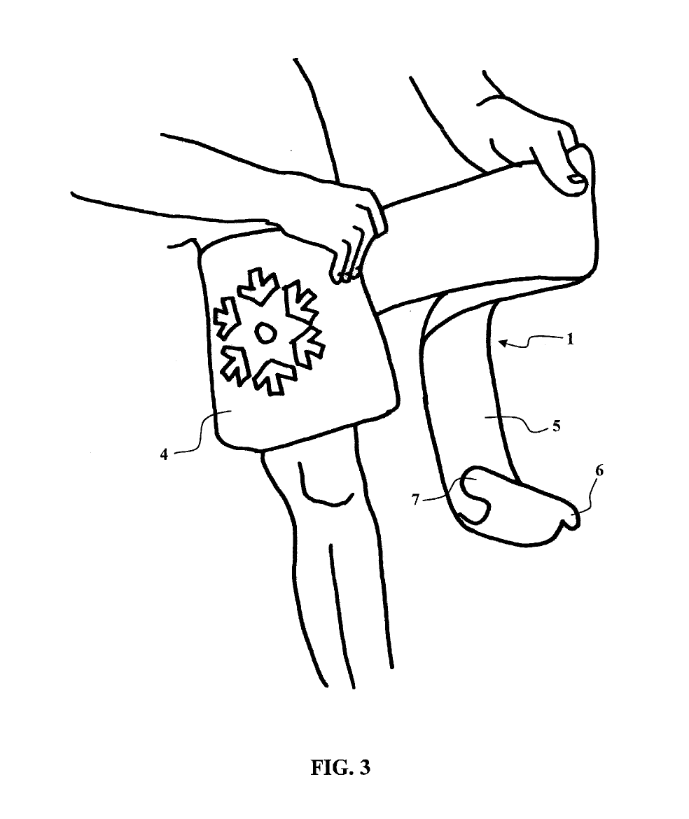

Reference will now be made to FIGS. 3 to 9, which illustrate the use of the device according to the invention in the case of treatment of the human body.

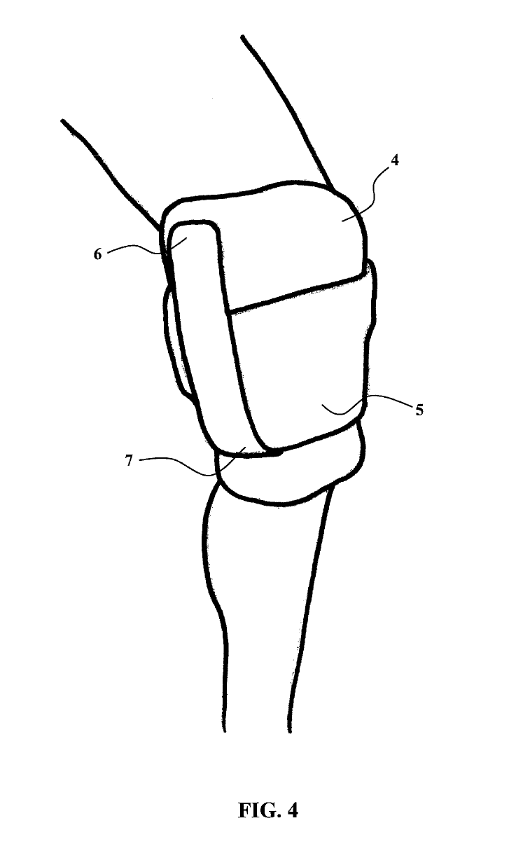

FIGS. 3 and 4 illustrate the use of the device around a knee of a patient. In FIG. 3, the receiving compartment 4 is placed on the injured area of the knee, and one starts winding the fixing portion 5 on. Naturally, the receiving compartment 4 bears on the injured area via its inner face provided with the wall 8 of netting. In FIG. 4, the winding on of the fixing portion 5 has been completed. The tongue 6 adheres to the outer surface of the receiving compartment 4, while the tongue 7 is folded back in order to engage under a part of the fixing portion 5.

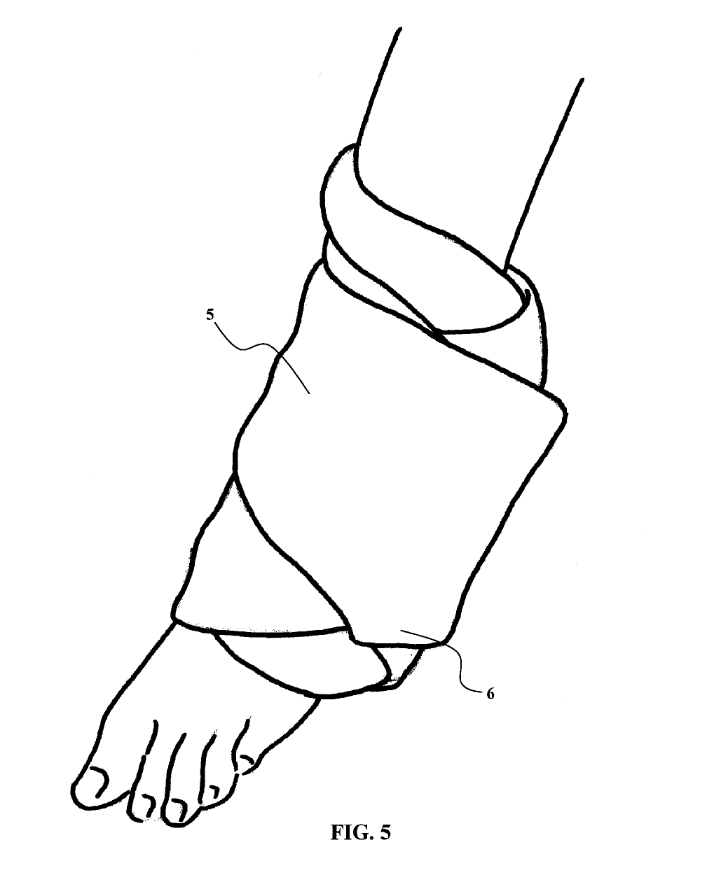

FIG. 5 illustrates the use of the device around the ankle of a patient. In this embodiment, the flexible bandage 1 has a single tongue 6 which, in FIG. 5, is folded back in order to engage under a part of the fixing portion 5.

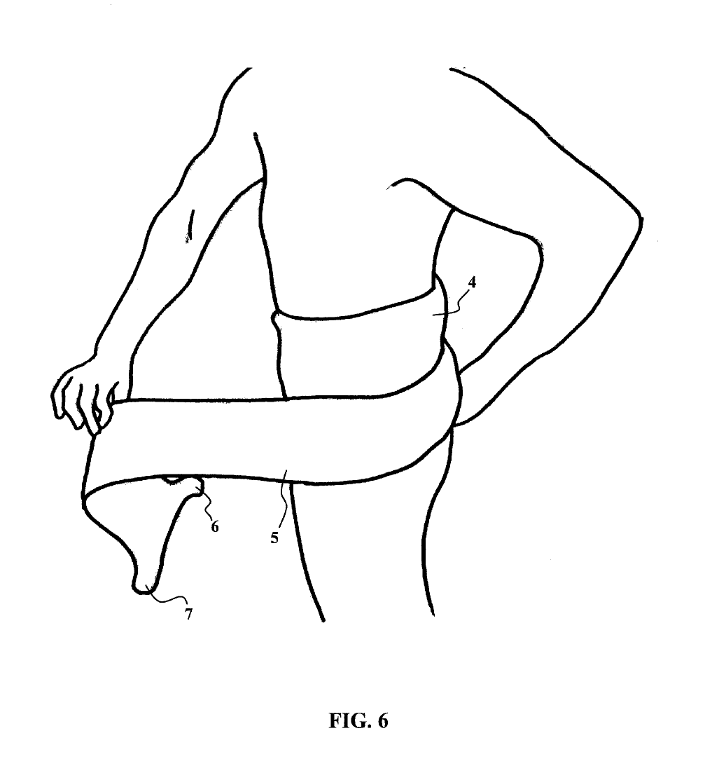

FIG. 6 illustrates the use of the device around the abdomen of a patient, for treatment of the back. The receiving compartment 4 is applied to the injured area of the back, and the fixing portion 5 is wound completely around the abdomen.

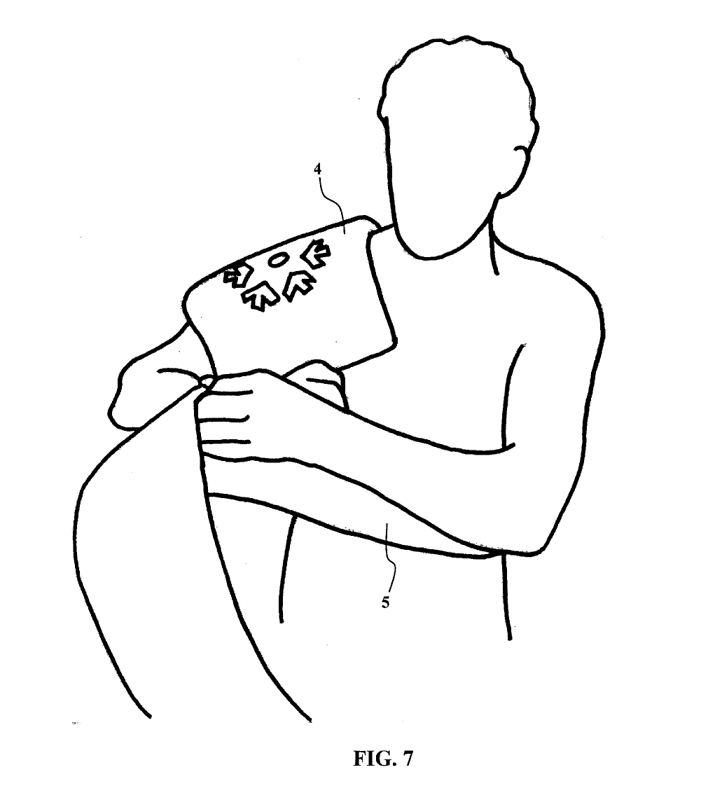

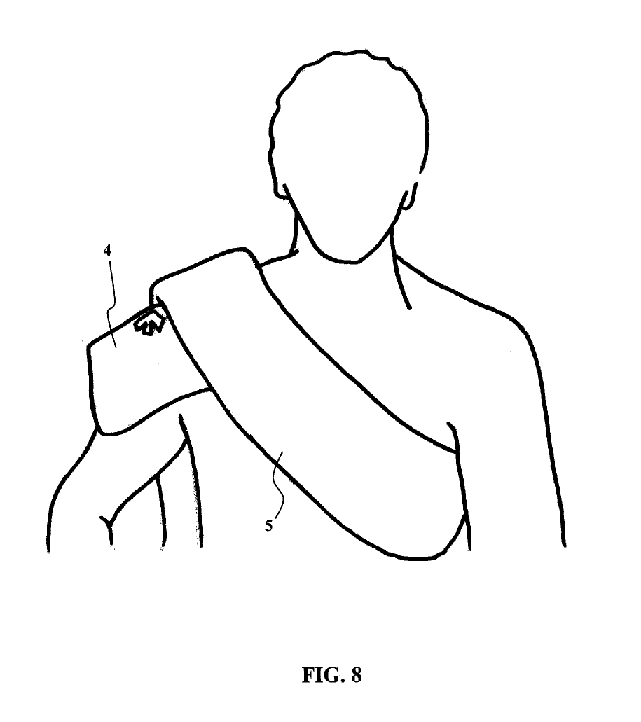

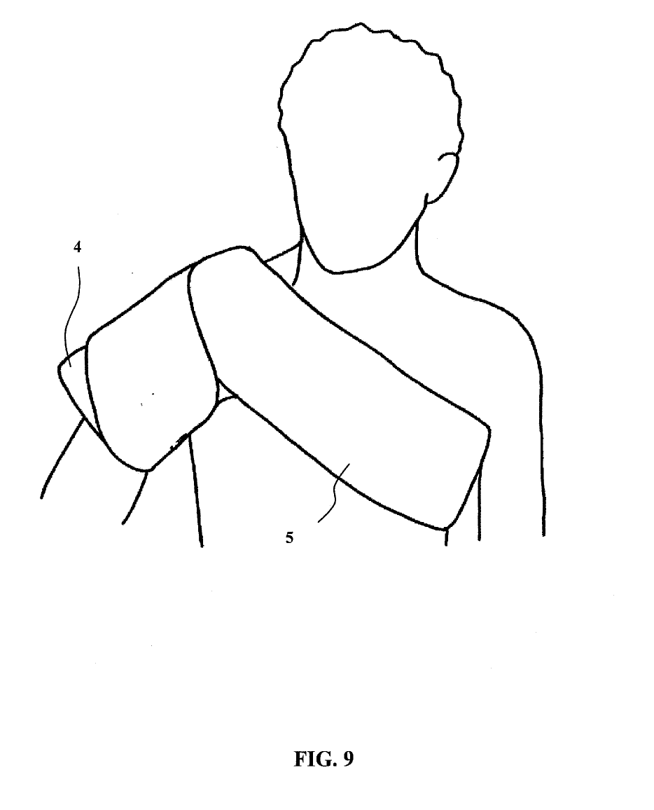

FIGS. 7 to 9 illustrate the use of the device for treating a shoulder of a patient. In FIG. 7, the receiving compartment 4 is placed over the shoulder, and one starts winding on the fixing portion 5 by passing it across the back, then under the left arm and across the front, such that it comes to bear on the receiving compartment 4. In FIG. 8, the fixing portion 5 passes over the receiving compartment 4 and descends across the back again near the right arm of the patient. In FIG. 9, the fixing portion 5 is then wound around the proximal area of the arm of the patient so as to bear on the receiving compartment 4, until complete winding.

The present invention is not limited to the embodiments that have been explicitly described, and instead it includes the variants and generalisations thereof that are contained within the scope of the attached claims.

1. A device for treatment by cold, comprising:
a flexible bandage (1) with a first face (1a) and second face (1b), having a receiving compartment (4) and a fixing portion (5),
an insert (9) which has a flexible pouch (9b) filled with a gel of thermal material (9c) able to maintain a reduction of the temperature, and which is shaped so as to be accommodated and retained in the receiving compartment (4),
wherein:
the flexible bandage (1) comprises a single fixing portion (5),
the receiving compartment (4) is arranged at a first end of the single fixing portion (5) of the flexible bandage (1),
the flexible bandage (1) is a self-adhesive bandage, of which the length (L) is greater than 1.0 m and which has self-fastening means (10, 11) distributed so as to permit adherence of any part of a substantial portion of its first face (1a) to any part of a substantial portion of its second face (1b).

2. The device for treatment by cold according to claim 1, wherein the flexible bandage (1) has a length (L) of between 1.0 m and 3.5 m, preferably of between 1.7 m and 1.9 m, for applications to the treatment of the human body.

3. The device for treatment by cold according to claim 2, wherein the fixing portion (5) of the flexible bandage (1) has a width (L3) of between 8 cm and 20 cm, preferably of between 12 cm and 15 cm.

4. The device for treatment by cold according to any claim 3, wherein the receiving compartment (4) is generally rectangular, with a width (L4) greater than the width (L3) of the fixing portion (5).

5. The device for treatment by cold according to claim 4, wherein the receiving compartment (4) has a width (L4) of between 24 cm and 30 cm, and a length (L2) of between 28 cm and 34 cm.

6. The device for treatment by cold according to claim 4, wherein the receiving compartment (4) is offset laterally with respect to the fixing portion (5), such that a first lateral edge (4a) of the receiving compartment (4) is in a continuation of a first lateral edge (5a) of the fixing portion (5).

7. The device for treatment by cold according to claim 1, wherein the flexible bandage (1) is made of a textile material having, on its first face (1a), a distributed structure with multiple micro-loops (10) and having, on its second face (1b) opposite the first face (1a), a distributed structure with micro-hooks (11) that are able to fasten themselves to said micro-loops (10).

8. The device for treatment by cold according to any claim 1, wherein the receiving compartment (4) is delimited, along an inner face (1b) of the flexible bandage (1), by a wall (8) of netting which is rigidly connected along its peripheral edge to the flexible bandage (1), except for a free edge (8a) defining an opening (4b) which is provided for passage of the insert (9) and which is oriented towards the fixing portion (5).

9. The device for treatment by cold according to claim 1, wherein the fixing portion (5) of the flexible bandage (1) has, at its second end (3), two transverse tongues (6, 7) extending away from each of its longitudinal edges (5a, 5b).

10. The device for treatment by cold according to claim 1, wherein the gel of thermal material (9c) is a gel based on propylene glycol maintaining its flexibility when cold or packaged in an insert having multiple compartments articulated with one another.

11. The device for treatment by cold according to claim 1, wherein the flexible pouch (9b) is made of silicone.
